SEQUENCE LISTING (4 sequences)

SEQ ID NO: 1 - RNA, 22 nt, Artificial Sequence
  miR-214-3p
acagcaggcacagacaggcagu

SEQ ID NO: 2 - RNA, 22 nt, Artificial Sequence
  miR-214-3p inhibitor
acugccugucugugccugcugu

SEQ ID NO: 3 - RNA, 19 nt, Artificial Sequence
  siLHX6 Sense
gagucauccuuuuucagua

SEQ ID NO: 4 - RNA, 19 nt, Artificial Sequence
  siLHX6 Antisense
uacugaaaaaggaugacuc

(19) United States
(12) Patent Application Publication
(10) Pub. No.: US 2022/0017971 A1
(43) Pub. Date: Jan. 20, 2022

(54) COMPOSITION FOR PREDICTING CHEMOTHERAPY RESISTANCE OF OVARIAN CANCER AND USE THEREOF

(71) Applicant: KOOKMIN UNIVERSITY INDUSTRY ACADEMY COOPERATION FOUNDATION, Seoul (KR)

(72) Inventors: Whasun Lim, Seoul (KR); Gwonhwa Song, Cheonan-si, Chungcheongnam-do (KR); Hee Seung Kim, Seoul (KR)

(73) Assignee: KOOKMIN UNIVERSITY INDUSTRY ACADEMY COOPERATION FOUNDATION, Seoul (KR)

(21) Appl. No.: 17/375,009

(22) Filed: Jul. 14, 2021

(30) Foreign Application Priority Data
Jul. 15, 2020 (KR) 10-2020-0087486
Jul. 5, 2021 (KR) 10-2021-0087674
Jul. 5, 2021 (KR) 10-2021-0087675

(51) Int. Cl. C12Q 1/6886; A61K 33/243; A61K 31/337; C12N 15/113

(52) U.S. Cl. CPC C12Q 1/6886; A61K 33/243; A61K 31/337; C12N 2310/14; C12Q 2600/178; C12N 2320/31; C12N 15/113

(57) ABSTRACT

The present invention relates to a composition for predicting resistance to chemotherapy for ovarian cancer and uses thereof, and more particularly, as the expression of exosome-derived miR-214-3p in epithelial ovarian cancer patients increases, the expression of the target gene LHX6 is suppressed, malignancy of epithelial ovarian cancer and anticancer drug resistance are increased, the change in the expression level of miR-214-3p and LHX6 can be provided as a composition for predicting the prognosis of cancer malignancy and anticancer drug resistance in epithelial ovarian cancer patients, and as it was confirmed that apoptosis of epithelial ovarian cancer cells treated with miR-214-3p inhibitors increases and the anticancer drug resistance is regulated by the expression regulation of miR-214-3p and LHX6, the miR-214-3p inhibitor and the activator or expression promoter of LHX6 may be provided as an anti-cancer therapeutic agent or an anti-cancer drug sensitizer for epithelial ovarian cancer cells.

### CROSS-REFERENCE TO RELATED APPLICATION

This application claims priority to and the benefit of Korean Patent Application No. 10-2020-0087486 filed in the Korean Intellectual Property Office on Jul. 15, 2020, Korean Patent Application No. 10-2021-0087674 filed in the Korean Intellectual Property Office on Jul. 5, 2021, and Korean Patent Application No. 10-2021-0087675 filed in the Korean Intellectual Property Office on Jul. 5, 2021, the entire contents of which are incorporated herein by reference.

### BACKGROUND OF THE DISCLOSURE

### 1. Field of the Disclosure

The present invention relates to a composition for predicting the prognosis of resistance to chemotherapy for ovarian cancer comprising exosome-derived miR-214-3p and LHX6, and to a use thereof.

### 2. Description of the Related Art

Most ovarian cancers originate from the ovarian and fallopian tube epithelium and have the lowest 5-year survival rate among gynecological malignancies. Epithelial ovarian cancer (EOC) is the fourth leading cause of female cancer-related deaths in Western countries, and its incidence is also increasing in Korea.

Since most ovarian cancers do not show symptoms even when they have metastasized to other organs, they are only detected at a fairly advanced stage, that is, at a high clinical stage. Therefore, ovarian cancer requires major surgery and at the same time a complex treatment method is required. Although ovarian cancer treatment involves surgical treatment followed by platinum-based chemotherapy, most patients experience cancer recurrence within 12 to 18 months after the first treatment. Therefore, understanding the molecular mechanisms behind the malignant transformation and recurrence of EOC and developing diagnostic markers to diagnose EOC patients are required.

Exosomes are small-sized vesicles responsible for intercellular interactions, and various types of proteins, miRNAs, mRNAs, lipids, etc. contained in the exosomes contain pathological characteristics, and thus are in the spotlight as a material for biomarkers for diseases. miRNA is a small non-coding RNA most abundantly present in exosomes, and it is predicted that various types of cancer can be diagnosed through miRNAs in exosomes derived from cancer cells.

### SUMMARY OF THE DISCLOSURE

The present invention predicts the resistance of chemotherapeutic agent for ovarian cancer by confirming the expression level of miR-214-3p and the target gene LHX6 whose expression is regulated by the miR-214-3p, and is intended to provide miR-214-3p and LHX6 expression modulators as a therapeutic agent for treating ovarian cancer and chemotherapy sensitizers

The present invention provides a composition for predicting prognosis of resistance to chemotherapy for ovarian cancer comprising at least one selected from the group consisting of miR-214-3p and LHX6.

The present invention provides a kit for predicting prognosis of resistance to chemotherapy for ovarian cancer, comprising an agent capable of detecting at least one selected from the group consisting of miR-214-3p and LHX6 from a non-invasive biological sample of blood or serum as an active ingredient.

The present invention provides a method of providing information necessary for predicting resistance of chemotherapy for a patient with ovarian cancer, comprising:

(a) measuring expression level of exosome-derived miR-214-3p in a non-invasive biological sample of blood or serum isolated from an ovarian cancer patient; and

(b) determining that the patient with ovarian cancer is resistant to chemotherapy when expression level of miR-214-3 in the sample of the (a) is higher than expression level of normal control compared to the expression level of the normal control.

The present invention provides a method of providing information necessary for predicting resistance of chemotherapy for a patient with ovarian cancer, comprising:

(a) measuring expression level of exosome-derived LHX6 in a non-invasive biological sample of blood or serum isolated from an ovarian cancer patient; and

(b) determining that the patient with ovarian cancer is resistant to chemotherapy when expression level of LHX6 in the sample of the (a) is lower than expression level of normal control compared to the expression level of the normal control.

The present invention provides a pharmaceutical composition for enhancing sensitivity of chemotherapy for ovarian cancer, comprising at least one selected from the group consisting of a miR-214-3p inhibitor, LHX6, an LHX6 activator and an LHX6 expression promoter as an active ingredient.

The present invention provides a pharmaceutical composition for preventing or treating ovarian cancer, which comprises at least one selected from the group consisting of a miR-214-3p inhibitor, LHX6, an LHX6 activator and an LHX6 expression promoter, and a chemotherapy, wherein the miR-214-3p inhibitor, LHX6, LHX6 activator or LHX6 expression promoter enhances therapeutic effect of the chemotherapy.

The present invention provides a method of screening a sensitizer for chemotherapy in treatment of ovarian cancer, comprising:

(a) measuring expression level of exosome-derived miR-214-3p or LHX6 obtained from blood or serum isolated from a patient with ovarian cancer;

(b) measuring expression level of exosome-derived miR-214-3p or LHX6 after treating a candidate material with blood or serum isolated from a patient with ovarian cancer; and

(c) determining that the candidate material is a sensitizer for chemotherapy when expression level of exosome-derived miR-214-3p in the (b) is lower than expression level of exosome-derived miR-214-3p in the (a), or expression level of LHX6 in the (b) is higher than expression level of LHX6 in the (a).

### BRIEF DESCRIPTION OF THE DRAWINGS

FIG. 1 shows a result of confirming the changes in expression of miRNAs based on ovarian tumor malignancy in the tissues of epithelial ovarian cancer patients with benign tumor (n=5), borderline serous tumor (n=4), low-grade serous ovarian cancer (LGSO, n=5), high-grade serous ovarian cancer (HGSO, PFI >12; n=5), partially platinum-sensitive high-grade serous ovarian cancer (HGSO, PFI 6-12; n=5) and platinum-resistant high-grade serous ovarian cancer (HGSO, PFI <6; n=5), and the expression of (A) miR-21-5p, (B) miR-141-3p, (C) miR-200a-3p, (D) miR-200b-3p, (E) miR-200c-3p, (F) miR-203-3p, (G) miR-205-5p, (H) miR-214-3p, and (I) miR-373-3p.

FIG. 2 shows a PCR analysis result of measuring the changes in expression of target miRNAs based on ovarian tumor malignancy in the tissues of epithelial ovarian cancer patients with benign tumor (n=5), borderline serous tumor (n=4), low-grade serous ovarian cancer (LGSO, n=5), high-grade serous ovarian cancer (HGSO, PFI >12; n=5), partially platinum-sensitive high-grade serous ovarian cancer (HGSO, PFI 6-12; n=5) and platinum-resistant high-grade serous ovarian cancer (HGSO, PFI <6; n=5), and the expression of (A) Rho GTPase activating protein 6 (ARHGAP6), (B) claudin 11 (CLDN11), (C) dual-specificity phosphatase 3 (DUSP3), (D) F-box protein 32 (FBXO32), (E) large tumor suppressor kinase 2 (LATS2), (F) LIM homeobox 6 (LHX6), (G) RAN-binding protein 6 (RANBP6), (H) suppressor of cytokine signaling 6 (SOCS6), and (I) transmembrane protein 170B (TMEM170B).

FIG. 3 shows a result of confirming changes in miRNA expression in exosomes derived from the blood of epithelial ovarian cancer patients with benign tumor (n=5), borderline serous tumor (n=4), low-grade serous ovarian cancer (LGSO, n=5), high-grade serous ovarian cancer (HGSO, PFI >12; n=5), partially platinum-sensitive high-grade serous ovarian cancer (HGSO, PFI 6-12; n=5) and platinum-resistant high-grade serous ovarian cancer (HGSO, PFI <6; n=5), and the expression of (A) miR-21-5p, (B) miR-141-3p, (C) miR-200a-3p, (D) miR-200b-3p, (E) miR-200c-3p, (F) miR-203-3p, (G) miR-205-5p, (H) miR-214-3p, and (I) miR-373-3p.

FIG. 4 shows a result of confirming effect of miR-214-3p inhibitor treatment on proliferation and death of epithelial ovarian cancer cell lines; (A) shows a result of confirming the decrease in LHX6 gene expression in OV90 cells according to the treatment of miR-214 mimic; (B) shows a result of confirming the decrease of LHX6 gene expression in ES2 cells according to the transfection of miR-214 mimic; (C) shows a result of confirming the increase of LHX6 gene expression in OV90 cells according to the treatment of miR-214-3p inhibitor in OV90 cells; (D) shows a result of confirming the increase of LHX6 gene expression in ES2 cells according to the treatment of miR-214-3p inhibitor in ES2 cells; (E) shows a result of confirming that the proliferation of OV90 cells and ES2 cells decreased according to the treatment with miR-214-3p inhibitor and the proliferation of OV90 cells and ES2 cells increased according to LHX6 inhibition; (F) shows a result of confirming the increase in mRNA expression level for PCNA protein essential for cellular proliferation in OV90 cells and ES2 cells according to LHLX6 inhibition; (G) shows a result of confirming the increase in the expression of PCNA protein in OV90 cells and ES2 cells according to LHX6 inhibition; and (H) shows a results of confirming the increase in apoptosis of OV90 cells and ES2 cells according to the treatment of the 214-3p inhibitor.

FIG. 5 shows a result of confirming the cell cycle, migration capabilities and mitochondrial function changes according to the miR-214-3p inhibitor treatment; (A) shows a result of confirming the increase in the ratio of SubG1 phase cells, which means the progression to apoptosis in the cell cycle of OV90 cells and ES2 cells, according to the treatment with the miR-214-3p inhibitor; (B) is a result confirming a decrease in the migration of OV90 cells and ES2 cells according to the treatment of miR-214-3p inhibitor, (C) shows a result of confirming the increase in ROS production, which means oxidative stress, in OV90 cells and ES2 cells according to the treatment with the miR-214-3p inhibitor; (D) shows a result of confirming the reduction in mitochondrial membrane potential in OV90 cells and ES2 cells according to the treatment with the miR-214-3p inhibitor; and (E) shows a result of confirming the increase in calcium ion (Ca²⁺) concentration in the OV90 cells and the ES2 cells according to the treatment with the miR-214-3p inhibitor.

FIG. 6 shows a result of confirming the change in PCNA protein expression and proliferative capacity according to LHX6 inhibition; (A) shows a result of confirming that the PCNA protein expression, which was decreased in OV90 cells and ES2 cells when cisplatin alone was treated, was restored according to LHX6 inhibition by observing the change in PCNA protein expression according to simultaneous cisplatin treatment and LHX6 inhibition; (B) shows a result of confirming that the PCNA protein expression, which was decreased in OV90 cells and ES2 cells when paclitaxel alone was treated, was restored according to LHX6 inhibition by observing the change in PCNA protein expression according to simultaneous paclitaxel treatment and LHX6 inhibition; and (C) shows a result of confirming the reduction in the proliferative capability of OV90 cells and ES2 cells when cisplatin or paclitaxel alone is treated, and the effect of confirming the proliferative recovery effect according to the additional inhibition of LHX6.

FIG. 7 shows a result of confirming the change in PCNA protein expression and proliferative capacity according to the miR-214-3p inhibitor treatment; (A) shows a result of confirming that the PCNA protein expression, which was decreased in OV90 cells and ES2 cells when cisplatin alone was treated, was further reduced by the treatment with the miR-214-3p inhibitor by observing the change in PCNA protein expression according to simultaneous cisplatin treatment and miR-214-3p inhibitor treatment; (B) shows a result of confirming that the PCNA protein expression, which was decreased in OV90 cells and ES2 cells when paclitaxel alone was treated, was further reduced by the treatment with the miR-214-3p inhibitor by observing the change in PCNA protein expression according to simultaneous paclitaxel treatment and miR-214-3p inhibitor treatment; and (C) shows a result confirming the synergistic effect on the reduction of the proliferative capability of OV90 cells and ES2 cells when treated with cisplatin or paclitaxel alone and the inhibition of proliferative capability by the combined treatment with the miR-214-3p inhibitor.

### DETAILED DESCRIPTION OF THE EMBODIMENTS

Hereinafter, the present invention will be described in more detail.

The inventors of the present invention confirmed that the overexpression of miR-214-3p and the expression of its target gene, LHX6, were suppressed in the exosomes identified from the body fluid of a patient with epithelial ovarian cancer and that inhibitors for miR-214-3p have shown an effect of inhibiting the proliferation of ovarian cancer cells and enhancing the apoptosis effect and completed the present invention.

The present invention may provide a composition for predicting the prognosis of resistance to chemotherapy for ovarian cancer, comprising at least one selected from the group consisting of miR-214-3p and LHX6.

The miR-214-3p may be derived from exosomes.

The miR-214-3p may be represented by the nucleotide sequence of SEQ ID NO: 1 (5′-ACA GCA GGC ACA GAC AGG CAG U-3′).

The LHX6 may be NCBI No. NM_001242333.

The chemotherapy may be an anticancer agent selected from the group consisting of cisplatin, carboplatin, oxaliplatin, paclitaxel and docetaxel.

The present invention may provide a kit for predicting the prognosis of resistance to chemotherapy for ovarian cancer, comprising an agent capable of detecting at least one selected from the group consisting of miR-214-3p and LHX6 from a non-invasive biological sample of blood or serum as an active ingredient.

The miR-214-3p may be derived from exosomes.

The agent may be any one of a primer or a probe that specifically binds to at least one selected from the group consisting of miR-214-3p and LHX6.

The kit may include instructions for measuring expression level of exosome-derived miR-214-3p and determining as being resistant to the chemotherapy for ovarian cancer when the expression is increased compared to normal control.

The kit may include instructions for measuring expression level of LHX6 and determining that the chemotherapy as being resistant to the chemotherapy for ovarian cancer when the expression is reduced compared to normal control.

The chemotherapy may be an anticancer agent selected from the group consisting of cisplatin, carboplatin, oxaliplatin, paclitaxel and docetaxel.

As used herein, the term “prognosis” refers to prediction of disease progression and recovery, and refers to a prospect or a preliminary evaluation. For the purposes of the present invention, prognosis means determining whether the treatment is successful, survival, recurrence, metastasis, drug reactivity, resistance or not in the subject after treatment of epithelial ovarian cancer.

As used herein, the term “primer” refers to a nucleic acid sequence having a short free 3′ hydroxyl group, capable of base pairing with a complementary template and serving as a starting point for template strand copying. The primer can initiate DNA synthesis in the presence of a reagent for polymerization (i.e., DNA polymerase or reverse transcriptase) and four different nucleoside triphosphates in an appropriate buffer and temperature. PCR conditions and lengths of sense and antisense primers may be appropriately selected according to techniques known in the art.

As used herein, the term “probe” refers to a nucleic acid fragment such as RNA or DNA corresponding to several bases to several hundred bases in length that can specifically bind to other than mRNA., and is labeled to determine the presence or absence of specific mRNA and expression level. The probe may be manufactured in the form of an oligonucleotide probe, a single-stranded DNA probe, a double-stranded DNA probe, an RNA probe, or the like. Suitable probe selection and hybridization conditions may be appropriately selected according to techniques known in the art.

The present invention may provide a method of providing information necessary for predicting resistance of chemotherapy for a patient with ovarian cancer, comprising (a) measuring expression level of exosome-derived miR-214-3p in a non-invasive biological sample of blood or serum isolated from an ovarian cancer patient; and

(b) determining that the patient with ovarian cancer is resistant to chemotherapy when expression level of miR-214-3 in the sample of the (a) is higher than expression level of normal control compared to the expression level of the normal control.

The present invention may provide a method of providing information necessary for predicting resistance of chemotherapy for a patient with ovarian cancer, comprising (a) measuring expression level of exosome-derived LHX6 in a non-invasive biological sample of blood or serum isolated from an ovarian cancer patient; and

(b) determining that the patient with ovarian cancer is resistant to chemotherapy when expression level of LHX6 in the sample of the (a) is lower than expression level of normal control compared to the expression level of the normal control.

The present invention may provide a pharmaceutical composition for enhancing sensitivity of chemotherapy for ovarian cancer, comprising at least one selected from the group consisting of a miR-214-3p inhibitor, LHX6, an LHX6 activator and an LHX6 expression promoter as an active ingredient.

The chemotherapy may be an anticancer agent selected from the group consisting of cisplatin, carboplatin, oxaliplatin, paclitaxel, and docetaxel.

The miR-214-3p inhibitor may be one that modulates LHX6.

The present invention may provide a pharmaceutical composition for preventing or treating ovarian cancer, which comprises at least one selected from the group consisting of a miR-214-3p inhibitor, LHX6, an LHX6 activator and an LHX6 expression promoter, and a chemotherapy, wherein the miR-214-3p inhibitor, LHX6, LHX6 activator or LHX6 expression promoter enhances therapeutic effect of the chemotherapy.

The chemotherapy may be an anticancer agent selected from the group consisting of cisplatin, carboplatin, oxaliplatin, paclitaxel and docetaxel.

The present invention may provide a method of screening a sensitizer for chemotherapy in treatment of ovarian cancer, comprising (a) measuring expression level of exosome-derived miR-214-3p or LHX6 obtained from blood or serum isolated from a patient with ovarian cancer;

(b) measuring expression level of exosome-derived miR-214-3p or LHX6 after treating a candidate material with blood or serum isolated from a patient with ovarian cancer; and

(c) determining that the candidate material is a sensitizer for chemotherapy when expression level of exosome-derived miR-214-3p in the (b) is lower than expression level of exosome-derived miR-214-3p in the (a), or expression level of LHX6 in the (b) is higher than expression level of LHX6 in the (a).

The miRNA and gene expression may be measured by any one method selected from the group consisting of reverse transcription-polymerase chain reaction (RT-PCR), quantitative PCR (RT-qPCR), Western blotting, and flow cytometry (FACS), but it is not limited thereto.

In one embodiment of the present invention, the pharmaceutical composition may be any one formulation selected from the group consisting of injections, granules, powders, tablets, pills, capsules, suppositories, gels, suspensions, emulsions, drops or liquids according to a conventional method.

In one embodiment of the present invention, the pharmaceutical composition may further comprise at least one additive selected from the group consisting of suitable carriers, excipients, disintegrants, sweeteners, coating agents, swelling agents, slip modifiers, flavors, antioxidants, buffers, bacteristats, diluents, dispersants, surfactants, binders and lubricants, which are conventionally used for the preparation of the pharmaceutical composition.

Examples of the carrier, excipient and diluent include lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate and mineral oil. Solid formulations for oral administration include tablets, pills, powders, granules, capsules, etc., and such solid formulations may contain at least one excipient such as starch, calcium carbonate, sucrose or lactose, gelatin and the like in addition to the composition. Furthermore, in addition to simple excipients, lubricants such as magnesium stearate and talc are also used. Examples of the liquid formulations for oral administration include suspensions, solutions, emulsions, syrups and the like, and various excipients such as wetting agents, sweeteners, fragrances, preservatives and the like may be included in addition to water and liquid paraffin which are commonly used as simple diluents. Formulations for parenteral administration include sterile aqueous solutions, non-aqueous solutions, suspensions, emulsions, freeze-dried preparations, suppositories and the like. Examples of the non-aqueous solution and the suspension include propylene glycol, polyethylene glycol, vegetable oil such as olive oil, injectable ester such as ethyl oleate, and the like. As the base of the suppository, witepsol, macrogol, tween 61, cacao butter, laurinum, glycerogelatin and the like can be used.

According to one embodiment of the invention, the pharmaceutical composition may be administered intravenously, intraarterially, intraperitoneally, intramuscularly, intrasternally, transdermally, intranasally, inhaled, topically, rectally, orally, intraocularlly or intradermally to the subject in the conventional manner.

The preferred dosage of the miR-214-3p expression inhibitor, LHX6, LHX6 activator or LHX6 expression promoter may vary depending on the condition and weight of the subject, the type and extent of the disease, the drug form, the route of administration, and the duration, and may be appropriately selected by those skilled in the art. According to one embodiment of the present invention, the daily dosage may be, but is not limited to, 0.01 to 200 mg/kg, specifically 0.1 to 200 mg/kg, more specifically 0.1 to 100 mg/kg. Administration may be administered once a day or divided into several times, and the scope of the invention is not limited thereto.

In the present invention, the ‘subject’ may be a mammal including a human, but it is not limited thereto.

Hereinafter, the present invention will be described in more detail through examples. These examples are only intended to illustrate the present invention in more detail, and it will be apparent to those skilled in the art that the scope of the present invention is not limited by these examples according to the gist of the present invention. The examples of the present invention are provided to more completely explain the present invention to those of ordinary skill in the art.

### Experimental Example

The following experimental examples are intended to provide experimental examples commonly applied to each embodiment according to the present invention.

1. Human Tissues and Serum Identification

Ovarian cancer tissues were collected from 29 patients with advanced ovarian cancer after primary debulking surgery and frozen in liquid nitrogen. The collected biopsies were benign serous cystadenoma (n=5), borderline serous tumor (n=4), low-grade serous ovarian cancer (LGSO; n=5), or high-grade serous ovarian cancer (HGSO; n=15). Further, the HGSO biopsies were divided according to their platinum-free interval (PFI), which is defined as the duration from the completion of platinum-based chemotherapy to recurrence. The PFI-based subclassification of HGSO tissues was as follows: platinum-sensitive (PFI >12 months; n=5); partial platinum-sensitive (6 months PFI <12 months; n=5); platinum-resistant (PFI <6 months; n=5).

Blood samples were collected from the same patients before surgery, and the peripheral blood was centrifuged to obtain matched serum and stored in liquid nitrogen. All tissues and blood samples were obtained from Seoul National University Hospital Human Biobank, and was approved by the Institutional Review Board (IRB) of Seoul National University Hospital (No. 1708-053-876).

2. Analysis for microRNA Expression

Total RNA of ovarian cancer tissues and epithelial ovarian cancer cells was extracted using Trizol reagent (Invitrogen). For cDNA synthesis for candidate microRNA expression analysis, a miRNA first-strand cDNA was synthesized using synthesis kit (Agilent Technologies). The miRNAs expression was measured using the High-Specificity miRNA QPCR Core Reagent Kit (Agilent Technologies, and the miRNA expression level was normalized based on the U6 snRNA expression level.

3. Analysis for mRNA Expression

cDNA was synthesized from 1 μg of RNA using AccuPower RT PreMix (Bioneer, Daejeon, Korea). Gene expression was measured using SYBR Green (Sigma) and the StepOnePlus Real-Time PCR system (Applied Biosystems, Foster City, Calif.). PCR conditions were incubated at 95° C. for 3 minutes, then 95° C. 30 seconds, 60° C. 30 seconds, and 72° C. 3 minutes conditions were amplified 40 times, and normalized based on the GAPDH expression level.

4. Exosome Extraction

To identify exosomes from the serum of epithelial ovarian cancer patients, Total Exosome Isolation Reagent (from serum) (Thermo Fisher Scientific, catalog number: 4478360) was used according to the manufacturer's instructions. In addition, to extract RNA from exosomes, Total Exosome RNA and Protein Isolation Kit (Thermo Fisher Scientific, catalog number: 4478545) was used according to the manufacturer's instructions.

5. Transfection

Epithelial ovarian cancer OV90 and ES2 cells were cultured in 6 wells, and a non-specific control and target oligonucleotide were transfected using Lipofectamine 2000 (Invitrogen). All oligonucelotides were synthesized by to Bioneer. Oligonucleotide and Lipofectamine 2000 were diluted in Opti-MEM reduced serum medium (Gibco, Cat No: 32985070) and then treated with cells. After 6 h of incubation at 37° C. in a 5% CO₂ incubator, the medium was replaced with a medium containing FBS.

6. Analysis of Cell Proliferation Capacity Using BrdU

An experiment was performed according to the manufacturer's manual using the BrdU kit (Cat No: 1167229001, Roche) to confirm the change in the proliferation ability of epithelial ovarian cancer cells. After 48 hours of incubation, 10 μM BrdU was additionally added to each well and incubated for 2 hours at 37° C./5% CO₂ in an incubator. After labeling the epithelial ovarian cancer cells with BrdU and fixing the cells, they were incubated with anti-BrdU-POD solution at room temperature for 90 minutes, and then washed three times. Finally, the cells were reacted with 100 μl of 3,3′, 5,5′-tetramethylbenzidine substrate, and the cell proliferation ability was analyzed by measuring the absorbance at 370 nm and 492 nm using an ELISA reader.

7. Cell Cycle Analysis Using Propidium Iodide Staining

In order to confirm the cell cycle change of ovarian cancer cells by the miR-214-3p inhibitor, 5×10⁵ cells were cultured in 6 wells until 70 to 80% abundance appeared. Thereafter, miR-214-3p inhibitors were treated in a dose-dependent manner (10, 20, 40 nM) and cultured in a 37° C./5% CO₂ incubator for 48 hours. Then, using trypsin, the cells were removed from the culture dish, washed with PBS, and the cells were slowly mixed using 1 mL of 1× binding buffer, and then centrifuged to obtain a cell pellet.

Next, the cells were suspended and cultured with 200 μL of 1× binding buffer, put in 100 μL in a brown 1.5 mL tube, and 5 μL of RNase and 5 μL of PI were mixed together to stain the cells for 1 hour at room temperature. After that, 400 μL of 1× binding buffer was added, the stained solution was transferred to a 5 mL FACS tube, and the fluorescence intensity was analyzed using a flow cytometer to measure the number of cells corresponding to each cell cycle.

8. Analysis of Apoptosis by Staining with Annexin V and Propidium Iodide

In order to confirm the change in the apoptosis effect of epithelial ovarian cancer cells, an experiment was performed using the FITC Annexin V cell death diagnostic kit I (BD Biosciences). Using trypsin, the cells were removed from the culture dish, washed with PBS, and the cells were slowly mixed using 1 mL of 1× binding buffer and then centrifuged to obtain a cell pellet.

Next, the cells were suspended and cultured with 200 μL of 1× binding buffer, put into 100 μL of a brown 1.5 mL tube, and cells were mixed with 5 μL of Annexin V and 5 μL of PI, and then stained at room temperature for 15 minutes. After that, 400 μL of 1×binding buffer was added and the stained solution was transferred to a 5 mL FACS tube, and then the fluorescence intensity was analyzed using a flow cytometer to measure the number of apoptotic cells.

9. Analysis of Calcium Concentration in Cells and Mitochondria by Fluo-4 and rhod-2 Staining

To check the intracellular calcium concentration of ovarian cancer cells, 3 μM of fluo-4 acetoxymethyl ester (AM) (Invitrogen) was used. First, 5×10⁵ cells were cultured in 6 wells. Thereafter, the miR-214-3p inhibitor was dose-dependently treated and cultured in a 37° C./5% CO₂ incubator for 48 hours. After that, cells were removed from the culture dish using trypsin, washed with PBS, and stained with 3 μM fluo-4 in an incubator at 37° C./5% CO₂ for 20 minutes, washed with PBS, and then the stained solution was transferred into a FACS tube and the fluorescence intensity was analyzed using a flow cytometer to determine the number of killed cells.

10. Immunofluorescence

Transfected epithelial ovarian cancer cells were fixed with methanol for 10 minutes and treated with PCNA antibody diluted in 1:100. After that, after washing twice with PBS containing 0.1% bovine serum albumin (BSA), the secondary antibody goat anti-mouse IgG Alexa 488 (catalog number: A11017, Invitrogen, Carlsbad, Calif.) was added to antibody dilution buffer so as to be diluted in 1:200 and incubated for 1 hour at room temperature.

After washing the cells with 0.1% BSA-PBS, DAPI staining was additionally performed to allow simultaneous observation of the target protein as well as the nucleus. After finishing the experiment, cells were observed and photographed using an LSM710 (Carl Zeiss, Thornwood, N.Y., USA) confocal microscope.

11. Measurement of Mitochondrial Membrane Potential by JC-1 Staining

JC-1 mitochondrial membrane potential (MMP) changes were measured using a mitochondria staining kit (Cat No: CS0390, Sigma-Aldrich). 5×10⁵ ES2 and OV90 cells were each aliquoted into 6 wells, and the cells were cultured until 70 to 80% abundance of the culture dish appeared. Thereafter, the miR-214-3p inhibitor was dose-dependently treated and cultured in a 37° C./5% CO₂ incubator for 48 hours. Thereafter, cells were collected from the culture dish using trypsin and centrifuged to obtain a cell pellet. Cells were dissolved in JC-1 staining solution and incubated for 20 minutes at 37° C./5% CO₂ incubator. The stained cells were centrifuged again, washed with 1×JC-1 staining buffer, and then fluorescence intensity was analyzed using a flow cytometer.

12. Intracellular ROS Measurement Using DCFH-DA

To confirm the effect of miR-214-3p inhibitor on ROS generation in ovarian cancer cell lines, 2′,7′-dichlorofluorescin diacetate (DCFH-DA, Sigma), which is converted to 2′,7′-dichlorofluorescin (DCF) and shows fluorescence in the presence of peroxide, was used. ES2 and OV90 cells were collected through trypsin and centrifuged to obtain a cell pellet. After washing once with PBS, 10 μM of DCFH-DA was incubated in an incubator at 37° C. for 30 minutes. Thereafter, the cells were washed twice with PBS, and a miR-214-3p inhibitor was added thereto and incubated in an incubator at 37° C. for 1 hour. Treated cells were washed again with PBS and analyzed for DCF fluorescence intensity using flow cytometry.

13. Statistical Analysis

For all experimental results, the mean and standard error were calculated using a statistical analysis system (SAS), and one-way ANOVA was performed. Significance tests were performed at the P<0.05 level.

### <Example 1> Analysis of miRNA Expression Change in Epithelial Ovarian Cancer Patient Tissue According to Degree of Malignancy

To analyze the expression of nine candidate miRNAs known to be related to the progression of ovarian cancer from ovarian cancer patient tissues, total RNA was extracted and miRNA PCR analysis was performed.

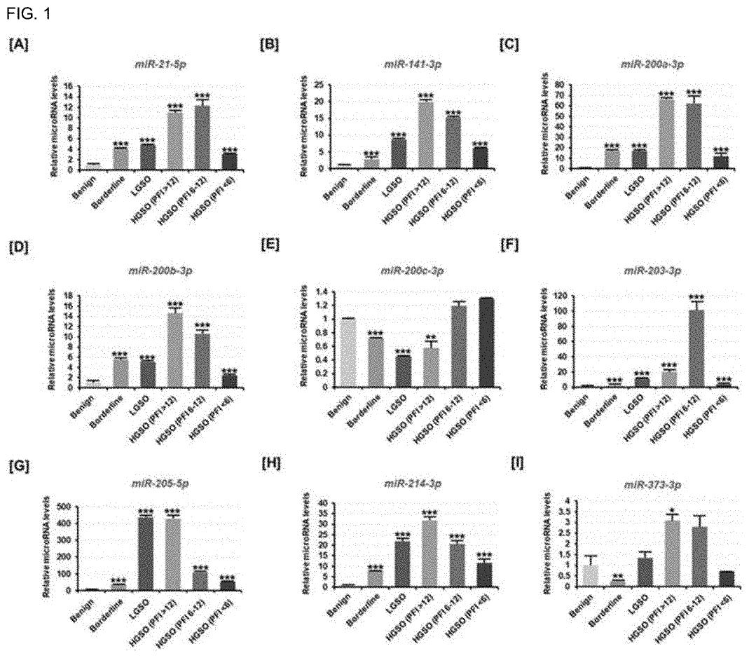

As a result, as shown in FIG. 1A to FIG. 1D and FIG. 1F to FIG. 1H, miRNA analysis revealed that the expression of miR-21-5p, miR-141-3p, miR-200a-3p, miR-200b-3p, miR-203-3p, miR-205-5p, and miR-214-3p was increased in borderline serous tumor, low-grade serous ovarian cancer (LGSO), and platinum-resistant high-grade serous ovarian cancer (HGSO) tissues compared to their expression in benign tumor. Notably, miR-214-3p expression was increased about 7.9-fold (p<0.001) in borderline tissue, 21.8-fold (p<0.001) in LGSO tissue, and 31.8-fold (p<0.001) in platinum-sensitive HGSO tissue compared to the miR-214-3p expression in benign tissue.

However, the expression change of miR-214-3p according to platinum resistance in high-grade serous ovarian cancer tissues was not clear. Conversely, the expression of miR-200c-3p was significantly reduced in tissues of patients with borderline tumors, low-grade serous ovarian cancer, and platinum-sensitive high-grade serous ovarian cancer. In addition, as shown in FIG. 1I, the differential expression of miR-373-3p with respect to ovarian tumor progression was difficult to determine.

Thus, the results suggested that the expression of miRNAs is significantly altered with respect to ovarian tumor progression and that they can be promoted as potential biomarkers.

Further, to analyze the expression change of the target gene of the candidate miRNA in the tissues of ovarian cancer patients, the genes which were reported to have tumor suppressor functions were selected among the target genes for the candidate miRNA through the miRNA database (http://mirdb.org).

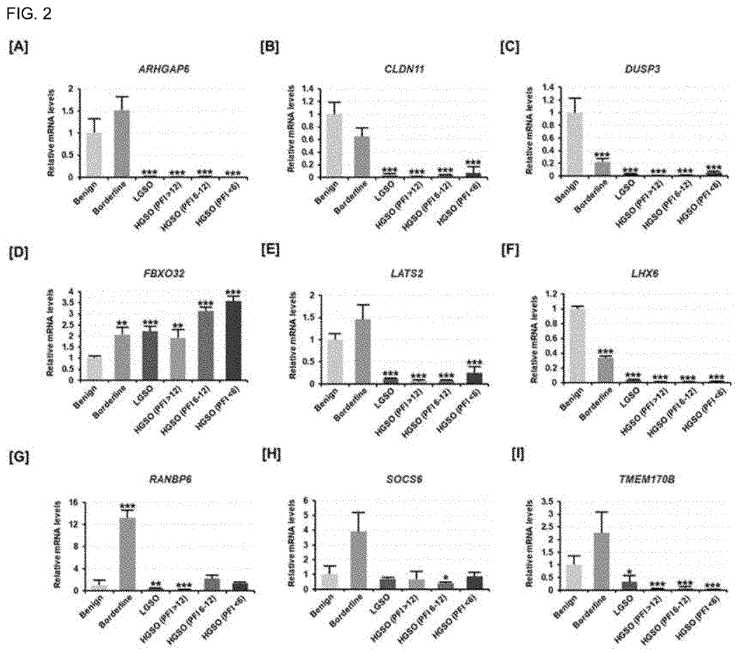

The PCR analysis revealed that the expression of target genes such as Rho GTPase activating protein 6 (ARHGAP6), claudin 11 (CLDN11), dual-specificity phosphatase 3 (DUSP3), large tumor suppressor kinase 2 (LATS2), LHX6, Ras-related nuclear protein (RAN)-binding protein 6 (RANBP6), suppressor of cytokine 6 (SOCS6), and transmembrane protein 170B (TMEM170B) tended to decrease in malignant tissues compared to their expression levels in benign tissues (FIG. 2A to FIG. 2C and FIG. 2E to FIG. 21). However, as shown in FIG. 2D, the expression of F-box protein 32 (FBXO32) gene was found to increase with ovarian tumor malignancy and drug resistance.

Thus, the results suggested that the candidate miRNAs and their respective target genes usually have opposite expression patterns.

### <Example 2> Analysis of miRNA Expression Changes in Exosomes Derived from Epithelial Ovarian Cancer Patient Blood According to Degree of Malignancy

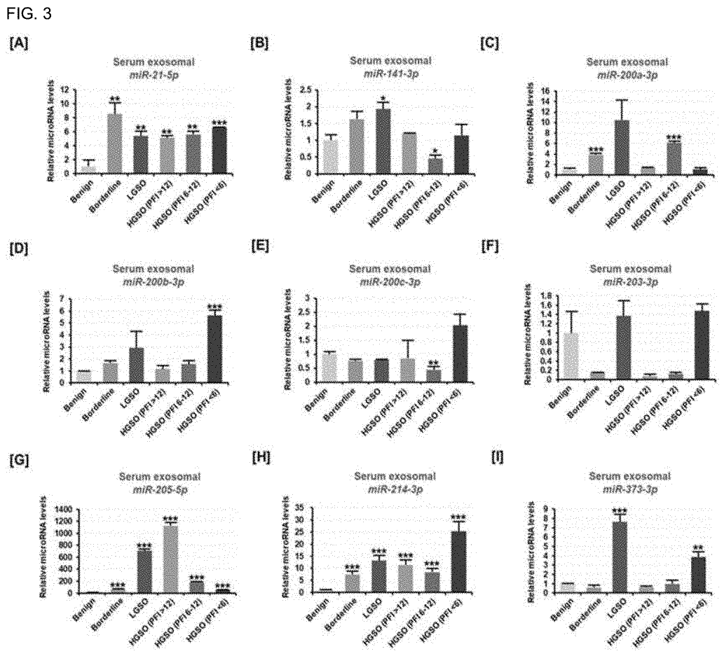

Further, the expression of candidate miRNAs was confirmed in exosomes derived from the serum of ovarian cancer patients. Referring to FIG. 3, the expression of miR-21-5p, miR-205-5p and miR-214-3p was significantly increased in serum-derived exosomes from patients with borderline tumors and malignant tumors. The expression of the remaining miRNAs (miR-141-3p, miR-200a-3p, miR-200b-3p, and miR-203-3p) was unclear with a tendency toward malignancy. Especially, miR-214-3p expression was significantly increased in exosomes derived from borderline (7.1-fold, p<0.001), LGSO (12.9-fold, p<0.001), platinum-sensitive HGSO (11.2-fold, p<0.001), partial platinum-sensitive HGSO (8.1-fold, p<0.001), and platinum-resistant HGSO (25.2-fold, p<0.001) compared to the positive control.

Based on the results, it can be suggested that miR-214-3p and its target gene, LHX6, can be utilized as biomarkers that can predict the malignancy of epithelial ovarian cancer.

### <Example 3> Functions to Regulate Proliferation and Death According to Regulation of miR-214-3p and LHX6 in Epithelial Ovarian Cancer Cells

Next, to analyze whether miR-214-3p can alter the characteristics of epithelial ovarian cancer cells, OV90 and ES2 cells, epithelial ovarian cancer cell lines, were transfected with a mimic for miR-214 and an inhibitor for miR-214-3p (SEQ ID NO: 2: 5′-ACU GCC UGU CUG UGC CUG CUG U-3′).

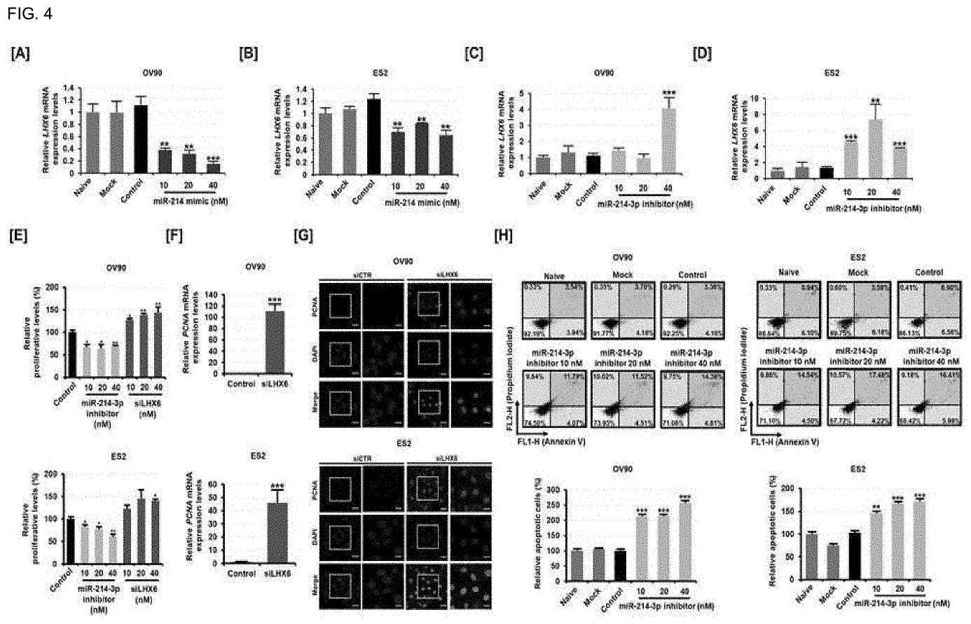

As a result, as shown in FIG. 4A to FIG. 4D, it was confirmed that the expression of the target gene LHX6 decreased according to miR-214 mimic transfection, whereas it increased according to the transfection for miR-214-3p inhibitor. In addition, it was confirmed that miR-214-3p inhibitor can inhibit the proliferative capability of OV90 and ES2 cells through BrdU incorporation analysis as shown in FIG. 4E. Inversely, siRNA that inhibits the expression of LHX6 (siLHX6, SEQ ID NO: 3: Sense 5′-GAG UCA UCC UUU UUC AGU A-3′, SEQ ID NO: 4: Antisense 5′-UAC UGA AAA AGG AUG ACU C-3′) was transfected, it was confirmed that the proliferative capacity of OV90 and ES2 cells was significantly increased. 40 nM of siLHX6 increased the proliferative levels of OV90 and ES2 cells by 1.4-fold.

In addition, as a result of analyzing the mRNA and protein level expression of PCNA, a protein essential for cell proliferation, through qPCR and immunofluorescence, respectively, as shown in FIG. 4F and FIG. 4G, it was confirmed that siLHX6 significantly increased the expression of PCNA in OV90 and ES2 cells.

Based on the results, it can be predicted that miR-214-3p can maintain the proliferation of EOC cells while LHX6 can decrease the proliferation. In addition, as result of analyzing the apoptosis pattern of OV90 and ES2 cells according to transfection of miR-214-3p inhibitor through annexin V and propidium iodide staining, apoptotic cell death was significantly increased when miR-214-3p inhibitor was transfected in OV90 and ES2 cells compared to the control group, as shown in FIG. 4H. The apoptotic OV90 and ES2 cells were found to increase 2.6-fold (p<0.001) and 1.7-fold (p<0.001), respectively, in response to 40 nM of miR-214-3p inhibitor.

### <Example 4> Analysis of Cell Properties Change by miR-214-3p Inhibitor in Epithelial Ovarian Cancer Cells

Next, miR-214-3p inhibitors were transfected into epithelial ovarian cancer cell lines, OV90 and ES2 cells.

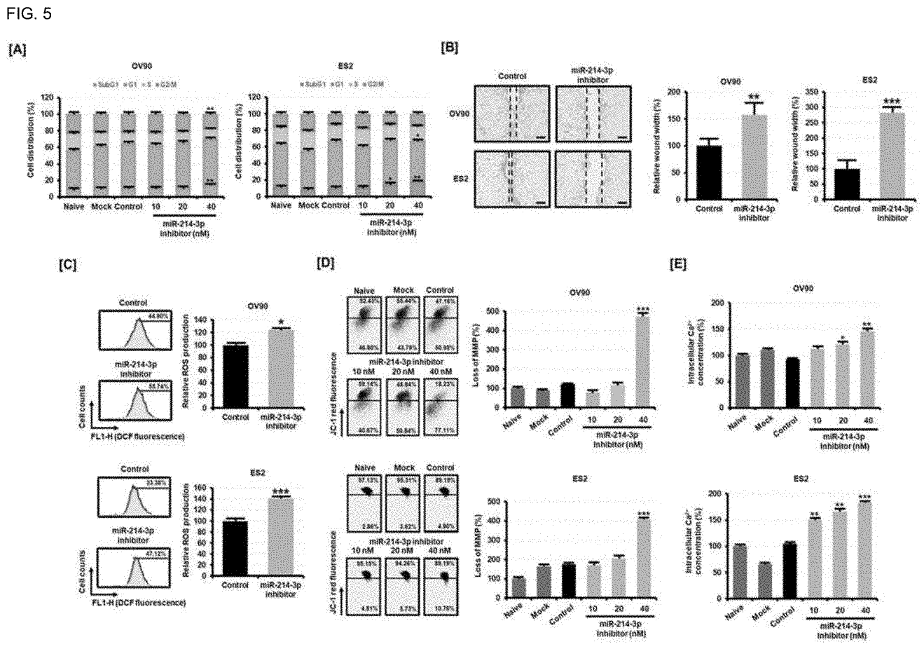

As a result, it was confirmed that apoptosis was induced by increasing sub-G1 of the cell cycle as shown in FIG. 5A and FIG. 5B, and cell migration was also reduced by the miR-214-3p inhibitor. In addition, as shown in FIG. 5C and FIG. 5E, it was confirmed that the transfection of miR-214-3p inhibitor increased reactive oxygen species in ovarian cancer cells, induced a change in mitochondrial membrane potential, increased intracellular calcium ions, and induced cell death.

### <Example 5> Analysis of Cell Proliferation Pattern by Combined Treatment with Anticancer Agent, LHX6 Inhibition, and miR-214-3p Inhibitor in Epithelial Ovarian Cancer Cells

Next, siLHX6 was transfected into epithelial ovarian cancer cell lines, OV90 and ES2 cells, and then, PCNA protein expression changes were analyzed by combined treatment with cisplatin or paclitaxel, which are standard anticancer drugs for ovarian cancer.

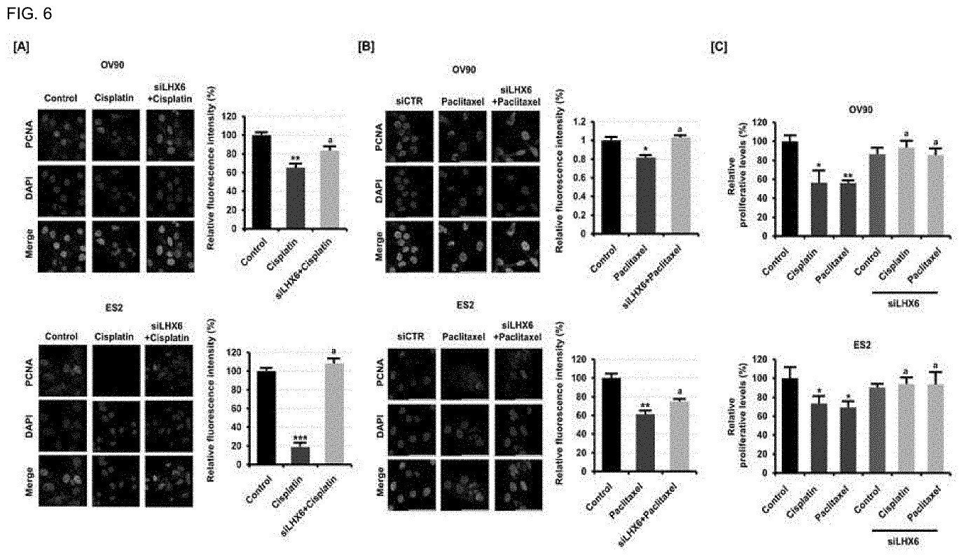

As a result, as shown in FIG. 6A and FIG. 6B, PCNA expression was significantly reduced by cisplatin in OV90 and ES2 cells, and transfection of siLHX6 restored PCNA expression reduced by cisplatin again.

In addition, BrdU incorporation analysis was performed to confirm whether transfection of siLHX6 actually restores the proliferative capacity of OV90 and ES2 cells. As a result, it was confirmed that siLHX6 transfection restored the proliferative capability of OV90 and ES2 cells reduced by cisplatin or paclitaxel treatment to the control level as shown in FIG. 6C.

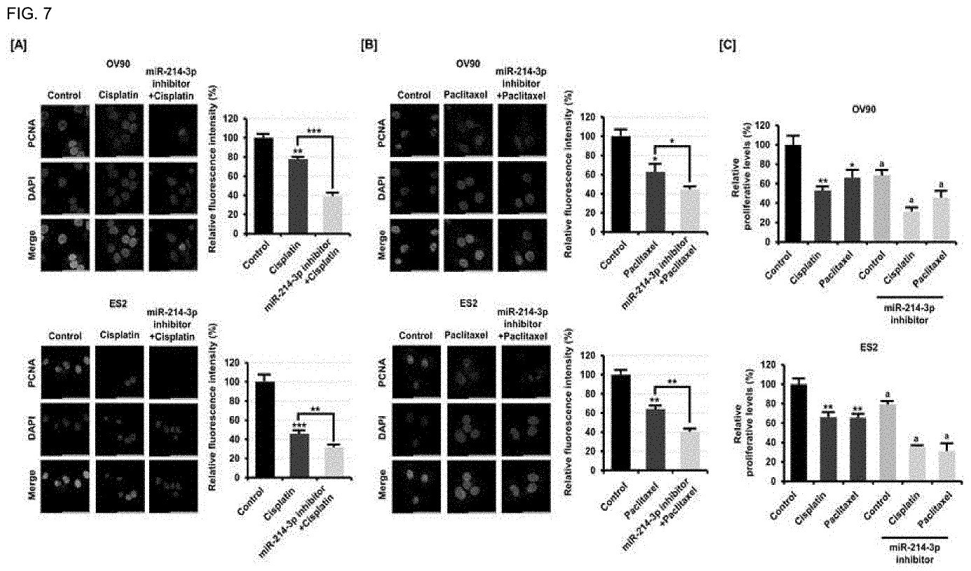

Next, it was confirmed whether transfection of miR-214-3p inhibitor could affect the proliferative capacity of epithelial ovarian cancer cell lines according to combined treatment with cisplatin or paclitaxel. Referring to FIG. 7A and FIG. 7B, it was confirmed that transfection of miR-214-3p inhibitor into OV90 and ES2 cells further decreased the expression of PCNA, which was reduced by treatment with cisplatin or paclitaxel. In addition, as shown in FIG. 7C, it was confirmed that miR-214-3p inhibitor transfection further reduced the proliferative capability of OV90 and ES2 cells, which were reduced by cisplatin or paclitaxel treatment.

From the above results, it was confirmed that the sensitivity of changes in cell proliferative capability to existing anticancer drugs was changed according to the regulation of LHX6 or miR-214-3p.

According to the present invention, as the expression of exosome-derived miR-214-3p in epithelial ovarian cancer patients increases, the expression of the target gene LHX6 is suppressed, malignancy of epithelial ovarian cancer and anticancer drug resistance are increased, the change in the expression level of miR-214-3p and LHX6 can be provided as a composition for predicting the prognosis of cancer malignancy and anticancer drug resistance in epithelial ovarian cancer patients, and as it was confirmed that apoptosis of epithelial ovarian cancer cells treated with miR-214-3p inhibitors increases and the anticancer drug resistance is regulated by the expression regulation of miR-214-3p and LHX6, the miR-214-3p inhibitor and the activator or expression promoter of LHX6 may be provided as an anti-cancer therapeutic agent or an anti-cancer drug sensitizer for epithelial ovarian cancer cells.

While the present invention has been particularly described with reference to specific embodiments thereof, it is apparent that this specific description is only a preferred embodiment and that the scope of the present invention is not limited thereby to those skilled in the art. That is, the practical scope of the present invention is defined by the appended claims and their equivalents.

What is claimed is:

1. A method of treating a patient with resistance to chemotherapy in ovarian cancer, comprising:
(a) measuring expression level of exosome-derived miR-214-3p in a non-invasive biological sample of blood or serum isolated from an ovarian cancer patient;
(b) determining that the patient with ovarian cancer is resistant to chemotherapy when expression level of miR-214-3 in the sample of the (a) is higher than expression level of normal control compared to the expression level of the normal control; and
(c) treating the patient with miR-214-3 inhibitor and chemotherapy.

2. A method of treating a patient with resistance to chemotherapy in ovarian cancer, comprising:
(a) measuring expression level of exosome-derived LHX6 in a non-invasive biological sample of blood or serum isolated from an ovarian cancer patient;
(b) determining that the patient with ovarian cancer is resistant to chemotherapy when expression level of LHX6 in the sample of the (a) is lower than expression level of normal control compared to the expression level of the normal control; and
(c) treating the patient with at least one selected from the group consisting of LHX6, an LHX6 activator and an LHX6 expression promoter, and chemotherapy.

3. A method for enhancing sensitivity of chemotherapy for ovarian cancer, comprising:
administering a pharmaceutical composition comprising at least one selected from the group consisting of a miR-214-3p inhibitor, LHX6, an LHX6 activator and an LHX6 expression promoter as an active ingredient to a patient.

4. The method for enhancing sensitivity of chemotherapy for ovarian cancer of claim 3, wherein the chemotherapy is an anticancer agent selected from the group consisting of cisplatin, carboplatin, oxaliplatin, paclitaxel and docetaxel.

5. The method for enhancing sensitivity of chemotherapy for ovarian cancer of claim 3, wherein the miR-214-3p inhibitor modulates LHX6.

6. A method for preventing or treating ovarian cancer, comprising:
administering a pharmaceutical composition comprising at least one selected from the group consisting of a miR-214-3p inhibitor, LHX6, an LHX6 activator and an LHX6 expression promoter, and a chemotherapy to a patient, wherein the miR-214-3p inhibitor, LHX6, LHX6 activator or LHX6 expression promoter enhances therapeutic effect of the chemotherapy.

7. The method for preventing or treating ovarian cancer of claim 6, wherein the chemotherapy is an anticancer agent selected from the group consisting of cisplatin, carboplatin, oxaliplatin, paclitaxel and docetaxel.

8. A method of screening a sensitizer for chemotherapy in treatment of ovarian cancer, comprising:
(a) measuring expression level of exosome-derived miR-214-3p or LHX6 obtained from blood or serum isolated from a patient with ovarian cancer;
(b) measuring expression level of exosome-derived miR-214-3p or LHX6 after treating a candidate material with blood or serum isolated from a patient with ovarian cancer; and
(c) determining that the candidate material is a sensitizer for chemotherapy when expression level of exosome-derived miR-214-3p in the (b) is lower than expression level of exosome-derived miR-214-3p in the (a), or expression level of LHX6 in the (b) is higher than expression level of LHX6 in the (a).
